(19) United States
(12) Patent Application Publication
(10) Pub. No.: US 2022/0091089 A1
(43) Pub. Date: Mar. 24, 2022

(54) APPARATUS AND METHODS FOR MEASURING SOIL CONDITIONS

(71) Applicant: AGCO Corporation, Duluth, GA (US)

(72) Inventor: Keith Robert CORPSTEIN, Beloit, KS (US)

(21) Appl. No.: 17/594,348

(22) PCT Filed: Mar. 17, 2020
(86) PCT No.: PCT/IB2020/052421

§ 371 Date: Oct. 12, 2021

Related U.S. Application Data
(60) Provisional application No. 62/838,555, filed on Apr. 25, 2019.

(51) Int. Cl. G01N 33/24; G01K 1/02; G01N 21/55; A01B 79/00; G01N 27/04

(52) U.S. Cl. CPC G01N 33/24; G01K 1/026; G01N 2033/245; A01B 79/005; G01N 27/04; G01N 21/55

(57) ABSTRACT

An apparatus for measuring a soil condition includes a frame coupled to a tow hitch and an instrumented shank engaged with the frame. The instrumented shank carries a plurality of sensors arranged such that each sensor is oriented to detect a soil property at a different depth than an adjacent sensor. A method of measuring a soil condition includes dragging an instrumented shank through soil. The instrumented shank carries a plurality of sensors. The method also includes detecting a soil property at a plurality of depths in the soil with the plurality of sensors.

### CROSS-REFERENCE TO RELATED APPLICATION

This application claims the benefit of the filing date of U.S. Provisional Patent Application 62/838,555, filed Apr. 25, 2019, the entire disclosure of which is incorporated herein by reference.

### FIELD

Embodiments of the present disclosure relate to measurement of soil conditions. More particularly, embodiments of the present invention relate to apparatus and methods for measuring soil conditions at different depths in conjunction with harvesting, tilling, or planting.

### BACKGROUND

Crop yields are affected by a variety of factors, such as seed placement, soil quality, weather, irrigation, and nutrient applications. Information about soil conditions is valuable because it assists farmers with determining how deep to plant seeds, how much water and fertilizer to apply, etc. Furthermore, crop yield can also be affected by soil conditions. Soil conditions can be improved by various techniques such as applying water or nutrients, tilling, etc. It is beneficial to know the conditions of soil before deciding what, if any, modifications to make to the soil or to a planting operation. For example, it would be beneficial for a farmer to have information about the soil conditions at each point in the field so that the field can be worked appropriately.

### BRIEF SUMMARY

An apparatus for measuring a soil condition includes a frame coupled to a tow hitch and an instrumented shank engaged with the frame. The instrumented shank carries a plurality of sensors arranged such that each sensor is oriented to detect a soil property at a different depth than an adjacent sensor. The soil property detected may include one or more of mean particle size, particle size distribution, soil pH, soil moisture, soil temperature, and residue content.

A method of measuring a soil condition includes dragging an instrumented shank through soil. The instrumented shank carries a plurality of sensors. The method also includes detecting a soil property at a plurality of depths in the soil with the plurality of sensors. The soil property detected may include one or more of mean particle size, particle size distribution, soil pH, soil moisture, soil temperature, and residue content.

### BRIEF DESCRIPTION OF THE DRAWINGS

While the specification concludes with claims particularly pointing out and distinctly claiming what are regarded as embodiments of the present disclosure, various features and advantages of embodiments of the disclosure may be more readily ascertained from the following description of example embodiments when read in conjunction with the accompanying drawings, in which:

FIG. 1 illustrates an apparatus for measuring a soil condition in accordance with one embodiment;

FIG. 2 illustrates a tractor pulling the apparatus of FIG. 1 in accordance with one embodiment;

FIG. 3 illustrates a method of measuring a soil condition in accordance with one embodiment, which may be used in conjunction with the apparatus shown in FIG. 1; and

FIG. 4 illustrates an example computer-readable storage medium comprising processor-executable instructions configured to embody one or more of the methods of operating the apparatus shown in FIG. 1, such as the method illustrated in FIG. 3.

### DETAILED DESCRIPTION

The illustrations presented herein are not actual views of any tillage implement or portion thereof, but are merely idealized representations that are employed to describe example embodiments of the present disclosure. Additionally, elements common between figures may retain the same numerical designation.

The following description provides specific details of embodiments of the present disclosure in order to provide a thorough description thereof. However, a person of ordinary skill in the art will understand that the embodiments of the disclosure may be practiced without employing many such specific details. Indeed, the embodiments of the disclosure may be practiced in conjunction with conventional techniques employed in the industry. In addition, the description provided below does not include all elements to form a complete structure or assembly. Only those process acts and structures necessary to understand the embodiments of the disclosure are described in detail below. Additional conventional acts and structures may be used. Also note, the drawings accompanying the application are for illustrative purposes only, and are thus not drawn to scale.

As used herein, the terms “comprising,” “including,” “containing,” “characterized by,” and grammatical equivalents thereof are inclusive or open-ended terms that do not exclude additional, unrecited elements or method steps, but also include the more restrictive terms “consisting of” and “consisting essentially of” and grammatical equivalents thereof.

As used herein, the term “may” with respect to a material, structure, feature, or method act indicates that such is contemplated for use in implementation of an embodiment of the disclosure, and such term is used in preference to the more restrictive term “is” so as to avoid any implication that other, compatible materials, structures, features, and methods usable in combination therewith should or must be excluded.

As used herein, the term “configured” refers to a size, shape, material composition, and arrangement of one or more of at least one structure and at least one apparatus facilitating operation of one or more of the structure and the apparatus in a predetermined way.

As used herein, the singular forms following “a,” “an,” and “the” are intended to include the plural forms as well, unless the context clearly indicates otherwise.

As used herein, the term “and/or” includes any and all combinations of one or more of the associated listed items.

As used herein, spatially relative terms, such as “beneath,” “below,” “lower,” “bottom,” “above,” “upper,” “top,” “front,” “rear,” “left,” “right,” and the like, may be used for ease of description to describe one element's or feature's relationship to another element(s) or feature(s) as illustrated in the figures. Unless otherwise specified, the spatially relative terms are intended to encompass different orientations of the materials in addition to the orientation depicted in the figures.

As used herein, the term “substantially” in reference to a given parameter, property, or condition means and includes to a degree that one of ordinary skill in the art would understand that the given parameter, property, or condition is met with a degree of variance, such as within acceptable manufacturing tolerances. By way of example, depending on the particular parameter, property, or condition that is substantially met, the parameter, property, or condition may be at least 90.0% met, at least 95.0% met, at least 99.0% met, or even at least 99.9% met.

As used herein, the term “about” used in reference to a given parameter is inclusive of the stated value and has the meaning dictated by the context (e.g., it includes the degree of error associated with measurement of the given parameter).

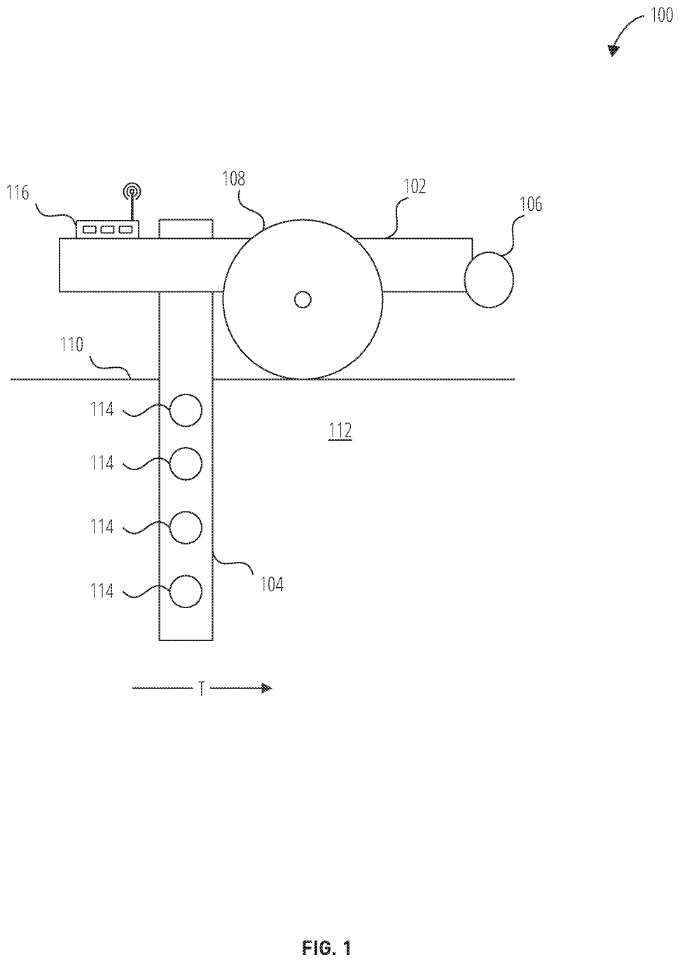

FIG. 1 is a simplified side view of an apparatus 100 for measuring a soil condition. In particular, the apparatus 100 may be used to measure conditions of the soil as a function of depth. The apparatus 100 includes a frame 102 and an instrumented shank 104 engaged with the frame 102. The frame 102 may be configured to be towed by a tractor or another vehicle via a tow hitch 106 coupled to the frame 102. The frame 102 may be at least partially supported by one or more wheels 108. The frame 102 is configured to travel in a direction T over a surface 110 of soil 112 in a field while the instrumented shank 104 passes through and cuts the soil 112. The instrumented shank 104 may be a beam oriented perpendicular to the frame 102, and may be fixed to the frame 102 by welds, bolts, or other attachment mechanisms. In certain embodiments, the instrumented shank 104 may carry a tilling tool, such as a plow, a knife, or other soil-working element. In some embodiments, the instrumented shank 104 may be removably or rotatably coupled to the frame 102, such that the frame 102 can travel over the surface 110 of the soil 112 without engaging the instrumented shank 104 with the soil 112 (e.g., when traveling over a roadway). The frame 102 may also carry other items, such as tillage implements, planting apparatus, weight, sensors, etc.

The instrumented shank 104 carries a plurality of sensors 114 at different points along the instrumented shank 104 to measure properties of the soil 112 at different depths. Though four sensors 114 are depicted, any number of sensors 114 may be present. In some embodiments, the sensors 114 are spaced between about 1 inch (2.54 cm) and about 6 inches (15.24 cm) from one another, measured center-to-center, such as between about 1 inch (2.54 cm) and about 2 inches (5.08 cm). For example, the sensors 114 may be spaced approximately 1.5 inches (3.81 cm) from one another. In some embodiments, the sensors 114 may be spaced such that they measure properties of different bands of the soil 112. For example, one sensor 114 may measure the soil 112 at depths between 0 inches (i.e., the surface 110) and 3 inches (7.62 cm), another sensor 114 may measure the soil 112 between 3 inches (7.62 cm) and 6 inches (15.24 cm), a third sensor 114 may measure the soil 112 between 6 inches (15.24 cm) and 12 inches (30.48 cm), and a fourth sensor 114 may measure the soil 112 between 12 inches (30.48 cm) and 18 inches (45.72 cm). The number and spacing of the sensors 114 may be selected based on expected field conditions and the resolution of data desired, among other considerations.

The sensors 114 may include any type or multiple types of sensor usable to measure properties of soil. For example, the sensors 114 may include optical sensors, reflectivity sensors, temperature sensors, electrical conductivity sensors, etc. The sensors 114 may be configured to detect properties such as organic material percentage in the soil 112, mean particle size, particle size distribution, soil pH (i.e., acidity), nitrogen concentration, soil moisture, soil temperature, and residue content. The detected properties may be selected such that differences in the detected properties can be used to distinguish different types of soil (e.g., topsoil vs. subsoil) and identify soil boundaries. The sensors 114 may typically be identical, but in some embodiments, one or more of the sensors 114 may different from other sensors 114. Sensors for detecting properties of soil are described generally in U.S. Patent Application Publication 2019/0075710, “Seed Trench Depth Detection Systems,” published Mar. 14, 2019; and U.S. Patent Application Publication 2018/0125002, “Systems, Methods, and Apparatus for Soil and Seed Monitoring,” published May 10, 2018; the entire disclosures of which are hereby incorporated herein by reference.

The frame 102 may also carry a transceiver 116 electrically connected to the sensors 114 and configured to receive signals from the sensors 114. The transceiver 116 may transmit the signals to another device, such as a computer within a tractor towing the apparatus 100, another vehicle, or a cellular network. The transceiver 116 may receive and transmit signals via wires or wirelessly. In some embodiments, the transceiver 116 may receive the signals from the sensors 114 via wires, and may transmit the signals to another device wirelessly.

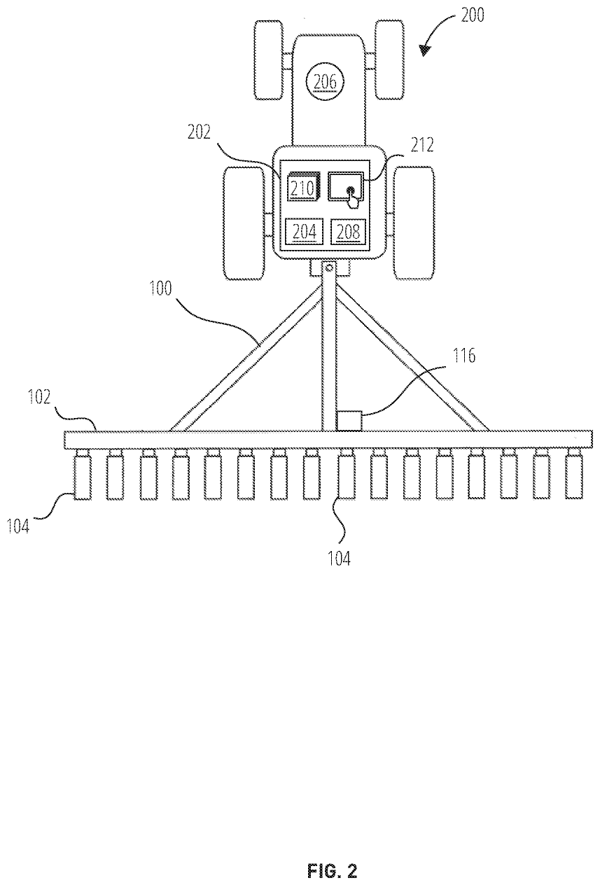

FIG. 2 is a simplified top view illustrating a tractor 200 drawing the apparatus 100, which includes the frame 102 supporting multiple shanks 104 and the transceiver 116. A computer 202, which may include a central processing unit (“CPU”), memory and graphical user interface (“GUI”) (e.g., a touch-screen interface), is located in the cab of the tractor 200. A global positioning system (“GPS”) receiver 206 may be mounted to the tractor 200 and connected to communicate with the computer 202. The computer 202 may include a processor 204 configured to communicate with the instrumented shank 104 and/or the GPS receiver 206, such as by wired or wireless communication.

The processor 204 may receive information from the transceiver 116 via a receiver 208 coupled to (e.g., within) the computer 202. The processor 204 may be configured to interpret the information, store the information in a data storage device 210, and/or display the information on a user interface 212 (depicted as a touch-screen, though other types of interface may also be used).

The computer 202 may correlate the information collected by the sensors 114 with a map of a field to provide data that an operator can use to make decisions about working the field. For example, the computer 202 may provide data relating the property of the soil 112 as a function of depth and location within the field. In some embodiments, the computer 202 may generate a moisture profile, a temperature profile, a nutrient profile, etc. Such profiles may be displayed on the user interface 212 in the tractor 200, may be viewed at a remote location, or may be saved and viewed after the tractor 200 has finished working the field.

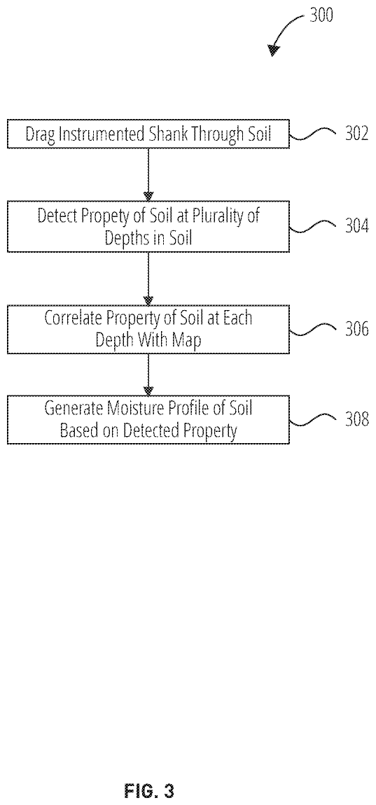

FIG. 3 is a simplified flow chart illustrating a method 300 in which the tractor 200 (FIG. 2) and the apparatus 100 (FIG. 1) may be used to work a field.

As depicted in block 302, the tractor 200 drags the instrumented shank 104 engaged with the frame 102 through soil 112. The instrumented shank 104 may cut through the soil, as opposed to simply traveling over the soil or in a trench formed by another object. The instrumented shank 104 carries a plurality of sensors 114.

In block 304, the sensors 114 detect a property of the soil 112 at a plurality of depths in the soil 112. For example, the sensors 114 may detect organic material percentage, particle size, soil pH, nitrogen concentration, moisture, temperature, residue, etc.

The method 300 may also include, as shown in block 306, correlating the property of the soil 112 measured by the sensors 114 at each depth with a map of the field in which the apparatus 100 operates. Thus, as shown in block 308, the computer 202 may generate a moisture profile or a profile of another property throughout the field.

In some embodiments, the apparatus 100 may also include a row unit, and the instrumented shank 104 may precede the row unit. That is, the tractor 200 may drag the instrumented shank 104 ahead of the row unit. An operating parameter of the row unit (e.g., a seed population, a seed depth, a down force, etc.) may be adjusted based on the property of the soil 112 detected by the sensors 114. In other embodiments, the apparatus 100 including the instrumented shank 104 may be used to identify field conditions for later planting. The apparatus 100 may be beneficially used to measure properties of a field shortly after harvest and/or shortly before replanting the field in a subsequent season.

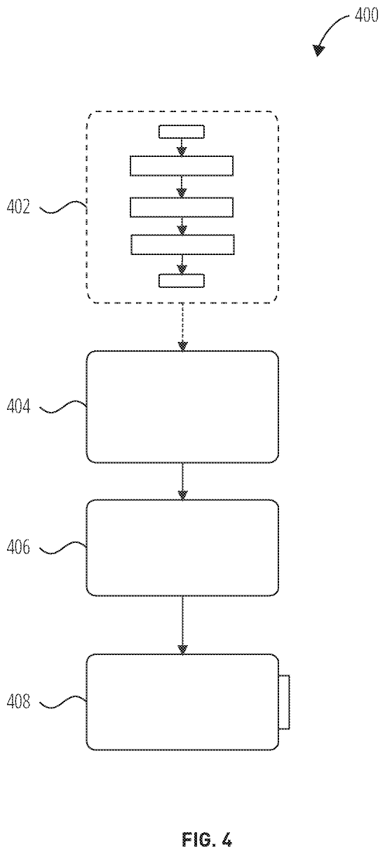

Still other embodiments involve a computer-readable storage medium (e.g., a non-transitory computer-readable storage medium) having processor-executable instructions configured to implement one or more of the techniques presented herein. An example computer-readable medium that may be devised is illustrated in FIG. 4, wherein an implementation 400 includes a computer-readable storage medium 402 (e.g., a flash drive, CD-R, DVD-R, application-specific integrated circuit (ASIC), field-programmable gate array (FPGA), a platter of a hard disk drive, etc.), on which is computer-readable data 404. This computer-readable data 404 in turn includes a set of processor-executable instructions 406 configured to operate according to one or more of the principles set forth herein. In some embodiments, the processor-executable instructions 406 may be configured to cause the computer 202 (FIG. 2) to perform operations 408 when executed via a processing unit, such as at least some of the example method 300 depicted in FIG. 3. In other embodiments, the processor-executable instructions 406 may be configured to implement a system, such as at least some of the example tractor 200 (FIG. 2) and apparatus 100 (FIG. 1). Many such computer-readable media may be devised by those of ordinary skill in the art that are configured to operate in accordance with one or more of the techniques presented herein.

The apparatus and methods disclosed herein may benefit a farmer by providing useful information about soil conditions with more detail than conventional processes. In particular, by providing a continuous measurement of properties as the apparatus 100 is towed through a field, and by measuring properties as multiple depths, a farmer can obtain a more complete view of how properties of the soil 112 vary throughout the field. This information can be used to make better decisions regarding how the field will be planted and worked. For example, adjustments may be made to seed population, seed depth, down force, water and nutrient application rates, etc.

Additional non-limiting example embodiments of the disclosure are described below.

Embodiment 1: An apparatus for measuring a soil condition, the apparatus comprising a frame coupled to a tow hitch, an instrumented shank engaged with the frame, and a plurality of sensors carried by the instrumented shank and arranged such that each sensor is oriented to detect a soil property at a different depth than an adjacent sensor. The soil property comprises at least one property selected from the group consisting of mean particle size, particle size distribution, soil pH, soil moisture, soil temperature, and residue content.

Embodiment 2: The apparatus of Embodiment 1, wherein the plurality of sensors comprises at least three sensors.

Embodiment 3: The apparatus of Embodiment 1 or Embodiment 2, wherein the plurality of sensors are spaced between 1 inch (2.54 cm) and 2 inches (5.08 cm) from adjacent sensors, measured center-to-center.

Embodiment 4: The apparatus of any one of Embodiment 1 through Embodiment 3, wherein the instrumented shank comprises a beam oriented perpendicular to the frame.

Embodiment 5: The apparatus of any one of Embodiment 1 through Embodiment 4, wherein the plurality of sensors comprises a plurality of optical sensors.

Embodiment 6: The apparatus of any one of Embodiment 1 through Embodiment 5, wherein the plurality of sensors comprises a plurality of reflectivity sensors.

Embodiment 7: The apparatus of any one of Embodiment 1 through Embodiment 6, wherein the plurality of sensors comprises a plurality of temperature sensors.

Embodiment 8: The apparatus of any one of Embodiment 1 through Embodiment 7, wherein the plurality of sensors comprises a plurality of electrical conductivity sensors.

Embodiment 9: The apparatus of any one of Embodiment 1 through Embodiment 8, further comprising a transceiver electrically connected to the plurality of sensors.

Embodiment 10: A system for measuring a soil condition, the system comprising the apparatus of any one of Embodiment 1 through Embodiment 11 and a receiver configured to receive information from the transceiver.

Embodiment 11: The system of Embodiment 10, further comprising a user interface configured to display data collected by the plurality of sensors.

Embodiment 12: The system of Embodiment 10 or Embodiment 11, further comprising a data storage device configured to store data collected by the plurality of sensors.

Embodiment 13: A method of measuring a soil condition, the method comprising dragging an instrumented shank through soil and detecting a soil property at a plurality of depths in the soil. The instrumented shank carries a plurality of sensors, and the soil property is detected with the plurality of sensors. The soil property comprises at least one property selected from the group consisting of mean particle size, particle size distribution, soil pH, soil moisture, soil temperature, and residue content.

Embodiment 14: The method of Embodiment 13, wherein dragging the instrumented shank comprises dragging the instrumented shank ahead of a row unit.

Embodiment 15: The method of Embodiment 14, further comprising adjusting an operating parameter of the row unit based on the soil property detected by the plurality of sensors.

Embodiment 16: The method of any one of Embodiment 13 through Embodiment 15, wherein dragging the instrumented shank comprises dragging the instrumented shank after harvesting a field and before replanting the field.

Embodiment 17: The method of any one of Embodiment 13 through Embodiment 16, wherein dragging the instrumented shank comprises cutting through the soil with the instrumented shank.

Embodiment 18: The method of any one of Embodiment 13 through Embodiment 17, further comprising correlating the soil property at each depth with a map of a field.

Embodiment 19: The method of any one of Embodiment 13 through Embodiment 18, further comprising generating a moisture profile of the soil based on the detected soil property.

While the present disclosure has been described herein with respect to certain illustrated embodiments, those of ordinary skill in the art will recognize and appreciate that it is not so limited. Rather, many additions, deletions, and modifications to the illustrated embodiments may be made without departing from the scope of the disclosure as hereinafter claimed, including legal equivalents thereof. In addition, features from one embodiment may be combined with features of another embodiment while still being encompassed within the scope as contemplated by the inventor. Further, embodiments of the disclosure have utility with different and various implement types and configurations.

1-12. (canceled)

13. A method of measuring a soil condition, the method comprising:
dragging an instrumented shank through soil ahead of a row unit, the instrumented shank carrying a plurality of sensors; and
detecting a soil property at a plurality of depths in the soil with the plurality of sensors, wherein the soil property comprises at least one property selected from the group consisting of mean particle size, particle size distribution, soil pH, soil moisture, soil temperature, and residue content.

14. (canceled)

15. The method of claim 13, further comprising adjusting an operating parameter of the row unit based on the soil property detected by the plurality of sensors.

16. The method of claim 13, wherein dragging the instrumented shank comprises dragging the instrumented shank after harvesting a field and before replanting the field.

17. The method of claim 13, wherein dragging the instrumented shank comprises cutting through the soil with the instrumented shank.

18. The method of claim 13, further comprising correlating the soil property at each depth with a map of a field.

19. The method of claim 13, further comprising generating a moisture profile of the soil based on the detected soil property.

20. A method of measuring a soil condition, the method comprising:
dragging an instrumented shank through soil after harvesting a field and before replanting the field, the instrumented shank carrying a plurality of sensors; and
detecting a soil property at a plurality of depths in the soil with the plurality of sensors, wherein the soil property comprises at least one property selected from the group consisting of mean particle size, particle size distribution, soil pH, soil moisture, soil temperature, and residue content.

21. The method of claim 20, wherein dragging the instrumented shank comprises cutting through the soil with the instrumented shank.

22. The method of claim 20, further comprising correlating the soil property at each depth with a map of a field.

23. The method of claim 20, further comprising generating a moisture profile of the soil based on the detected soil property.
